(19) United States
(12) Patent Application Publication
(10) Pub. No.: US 2008/0046078 A1
(43) Pub. Date: Feb. 21, 2008

(54) Silicone based ocular prosthesis, and method for making same

(76) Inventor: Matthew A. Singer, Memphis, TN (US)

Correspondence Address: BAKER, DONELSON, BEARMAN, CALDWELL & BERKOWITZ, PC, 900 SOUTH GAY STREET, 2200 RIVERVIEW TOWER, KNOXVILLE, TN, 37902, US

(21) Appl. No.: 11/607,227

(22) Filed: Dec. 1, 2006

Related U.S. Application Data
(60) Provisional application No. 60/822,447, filed on Aug. 15, 2006.

(51) Int. Cl. A61F 2/14; C08J 5/00; B44C 1/165; C04B 41/00; B29C 33/40; B29C 33/76

(52) U.S. Cl. 623/6.64; 264/222; 264/331.11; 264/132; 156/230; 264/317

(57) ABSTRACT

The present invention provides a silicone-based ocular prosthesis. In one aspect, the prosthesis includes a posterior sclera portion fabricated from a white-tinted silicone material. The sclera portion is formed from a molding process. The prosthesis also includes an anterior iris portion placed on the sclera portion. The prosthesis further includes a transparent layer over the sclera portion and the iris portion to provide a protective coating, and depth to the eye. Finally, a clear coat finish may optionally be applied to the prosthesis to create a realistic, wet look. A method for making the silicone-based ocular prosthesis using a molding process is also provided herein.

### STATEMENT OF RELATED APPLICATIONS

This application claims the benefit of U.S. Provisional Patent Application No. 60/822,447, filed Aug. 15, 2006. That application is entitled “Silicone Based Ocular Prosthesis and Method for Making the Same.” The provisional application is incorporated herein in its entirety by reference.

### FIELD OF THE INVENTION

The invention relates to an ocular prosthesis. More specifically, the invention relates to a flexible ocular prosthesis made of biocompatible, silicone-based materials. The invention also relates to methods for making a silicone-based ocular prosthesis.

### BACKGROUND OF THE INVENTION

Severe traumas and congenital abnormalities may cause the loss of a person's natural eye. Further, diseases such as infections tumors and glaucoma may necessitate the removal of a patient's natural eye. In such circumstances, the patient may desire or even require an ocular prosthesis. A well-formed ocular prosthesis can restore the anatomical structure of the eye and repair the cosmetic defect caused by injury or disease.

An ocular prosthesis generally comprises a scleral region (the white part of an eye ball). A life-like sclera will include veins, an iris, and a generally darker central pupil region. The iris preferably includes a color that replicates the patient's natural eye color. Some ocular prostheses may also comprise a clear corneal layer that gives the structure a bit of depth as well as a shiny appearance.

Early prior art ocular prosthetics used a glass object that was placed into the patient's orbital region. Ideally, the glass object had an iris painted thereon to simulate the person's other “natural” eye. More recently, and generally after World War II, the use of glass eyes was replaced by a hardened synthetic eye. Typically, the synthetic eye is fabricated from methylmethacrylate. Methylmethacrylate, or “MMA,” remains the material of choice for today's ocularist.

After a hardened replacement eye is fabricated, the iris and pupil are generally painted on the final prosthesis. A polymethyl methacrylate acrylic (PMMA) disc is painted to create an iris, and a PMMA corneal-pupil piece (CPP) that approximates the clear cornea is then adhered to the painted surface. Some consider the PMMA paint and the methymethacrylate monomers to be toxic to the human body. Caution must therefore be taken by the ocularist to avoid breathing in fumes from the monomers. Care must also be taken to avoid skin contact during the fabrication process.

Whether a replacement eye is made of glass or a hardened polymeric material, the replacement eye may not conform to the true shape of the patient's eye socket. Further, it can be painfull for the patient to receive the replacement eye, or to remove it as the replacement eye must be forced into the patient's orbit. Still further, a hardened replacement eye can be uncomfortable as it is non-compliant with the patient's muscular and tissue structure within the eye socket.

It is also noted that the hardened material used in present day replacement eyes are polished by removing material. This can create microscope voids or vugs in which undesirable biologic material might reside. Voids created during the shaping and polishing process may host harmful microorganisms that can be the source of infections.

Another drawback to the current hardened replacement eyes is that the polymerization process requires long curing times to ensure completion and to minimize the amount of trace monomers remaining in the final product. Some have suggested that outgassing of the PMMA materials can cause damage to the liver or the immune system of the patient. In addition, the PMMA polymer may also break down, creating toxic monomers and causing irritation and damage to the patient's tissue. The polymeric materials may also be unsuitable for certain patients simply due to a lack of biocompatibility.

PMMA is hard and inflexible. As such, prosthetics fabricated from PMMA are uncomfortable to wear by the patient, and may cause tearing and scarring inside the patient's eyelid and orbital tissue. This, in turn, may lead to infection and other damages to the patient.

Accordingly, there is a need for an improved ocular prosthesis and a method for making the same that overcome the above shortcomings of the prior art. A need further exists for an ocular prosthetic fabricated from a flexible yet biocompatible material. Still further, a need exists for an ocular prosthetic that is fabricated from silicone-base materials.

### SUMMARY OF THE INVENTION

The present invention provides a silicone-based ocular prosthesis. In one aspect, the prosthesis includes a posterior sclera portion fabricated from a white-tinted silicone material. The sclera portion is formed from a molding process. The prosthesis also includes an anterior iris portion placed on the sclera portion. The prosthesis further includes a transparent layer over the sclera portion and the iris portion to provide a protective coating, and depth to the eye. Finally, a clear coat finish may optionally be applied to the prosthesis to create a realistic, wet look.

The iris portion comprises a disk on the sclera portion. Preferably, the iris portion further comprises a centrally-located pupil portion formed from a depression in the disk. The depression is filled with a black-tinted silicone paint. The iris portion may be manually painted using silicone-based paints to create an appearance of a natural eye. Alternatively, the iris portion may be created by printing a digital image in the appearance of a natural iris.

The sclera portion of the ocular prosthesis may be solid, or it may have a hollow portion. Preferably, the iris portion and the sclera portion are integrally formed through painting rather than being formed via lamination or adherence of a disc.

A method for making an ocular prosthesis for a patient is also provided herein. In one embodiment, the method includes the step of filling a mold with a substantially white-tinted silicone material. The silicone material is allowed to cure to form a sclera portion of the ocular prosthesis. The method also includes placing an iris portion onto the sclera portion to form the ocular prosthesis. Preferably, the patient is a human, although the method may be used to provide an ocular prosthesis for either a live or a stuffed animal. In either instance, the method may further comprise inserting the ocular prosthesis into the eye socket of the patient.

The silicone material preferably comprises a whitening pigment suspended in a silicone oil. The silicone oil may be, for example, a 50-centistoke to a 1,000-centistoke oil. This creates a “Sclera Silicone”for the prosthesis. The silicone material may be a medical grade silicone. Alternatively, the silicone material may be a prosthesis grade silicone or other type of silicone.

The sclera portion may be painted in order to create natural details for the ocular prosthesis. The natural details may represent at least one of blood vessels, the limbus, and pink-shaded corner areas.

The step of placing an iris portion onto the sclera portion may be conducted by manually painting an iris onto the sclera portion. Alternatively, the step of placing an iris portion onto the sclera portion may be conducted by placing a colored disk onto the sclera portion. In one aspect, the colored disk is a printout of a digital image of an iris having a pupil.

The mold may be prepared in different ways. In one aspect, the mold is prepared by making an impression of the eye socket of the patient, and then preparing the mold based on the impression. In another aspect, the mold is prepared by taking a previously existing ocular prosthesis of the patient, and then preparing the mold based on the previously existing ocular prosthesis. In either event, the mold generally comprises, in one embodiment, a posterior portion corresponding to the sclera portion of the prosthesis, and an anterior portion corresponding to the iris portion of the prosthesis.

In preparing the prosthesis, the method may further include placing a thin layer of coring material within the posterior portion of the mold. The coring material serves to reserve a surface area of the sclera portion. The coring material is preferably a sulfur-free material. Non-limiting examples include a sulfur-free clay or a sulfur-free wax. An optional additional step is to place a vacuum on the substantially white-tinted silicone material to remove air bubbles prior to filling the mold.

Prior to the step of filling the mold with a clear silicone preparation, the sclera portion, the iris portion, or both, may be painted to create natural details for the ocular prosthesis. These may include blood vessels, the limbus, and pink-shaded corner areas. In addition, the method may also include the steps of removing the coring material from the mold, at least partially filling the mold with an optically clear silicone preparation, placing the sclera portion back into the mold, closing the mold, and then allowing the clear silicone preparation to cure. The step of allowing the clear silicone preparation to cure is preferably performed at an elevated temperature. In this way, a transparent layer is formed over the prosthesis. The transparent layer is formed from a clear silicone that fills the space in the mold previously occupied by the coring material. The transparent layer provides a protective silicone layer over the iris portion. The transparent layer may also help to prevent fading or color change to the iris portion over time. Still further, the transparent layer provided depth to the prosthesis.

The molding process may create a solid sclera portion. Alternatively, the sclera portion may have a hollow portion.

Optionally, a disk-shaped impression is placed on the thin layer of coring material before the Sclera Silicone is poured. The disk-shaped impression is located to correspond to the iris portion to be placed on the sclera portion. Preferably, the size of the disk-shaped impression corresponds to the size of an iris for the patient. In one optional aspect, the disk-shaped impression creates a slightly elevated profile for the disk in the forward or posterior direction.

In another aspect of the method, the step of removing a center portion of the elevated disk is provided. This creates a depression that corresponds to the size and shape of a pupil in the iris portion. The step of placing an iris portion onto the sclera portion may further comprise filling the depression with black silicone paint to create the pupil in the iris portion. The step of placing an iris portion onto the sclera portion may by performed by manually painting the iris portion around the pupil. The paint is preferably a silicone-based paint.

The method may also include the step of applying a polish coat to the ocular prosthesis. The polish coat creates a glossy and transparent appearance to the prosthesis. The polish coat would be applied before the ocular prosthesis is inserted into the eye socket of a patient. The ocularist or other practitioner may also choose to remove surface irregularities from the cured silicone preparation of the ocular prosthesis.

In one preferred embodiment, the method comprises providing an impression of an eye socket or an existing ocular prosthesis of the patient; preparing a mold based on the impression or the existing ocular prosthesis of the patient, wherein the mold comprises a posterior portion corresponding to the sclera portion of the ocular prosthesis, and an anterior portion corresponding to the iris portion of the ocular prosthesis; filling the anterior portion of the mold with a thin layer of clay, and creating a new, smooth surface on the clay inside the anterior portion; forming a disk-shaped impression on the thin layer of clay at a location corresponding to the iris to be formed on the ocular prosthesis, wherein the size of the disk-shaped impression corresponds to the desired size of the iris; forming the sclera portion of the ocular prosthesis by filling the mold with a white-tinted silicone preparation and allowing the silicone preparation to cure under suitable conditions, wherein the sclera portion so formed comprises an elevated thin disk corresponding to the location of the iris; removing a center portion of the disk-shaped impression to create a depression that corresponds to the size and shape of the pupil; filling the depression with black silicone preparation to create the pupil, and painting the sclera and the iris to create a desired appearance forming a semi-finished ocular prosthesis which is placed back into the posterior portion of the mold; removing the thin layer of clay from the anterior portion of the mold; filling the anterior portion of the mold with a clear silicone preparation; and allowing the silicone preparation to cure within the mold to form a transparent layer.

### BRIEF DESCRIPTION OF THE DRAWINGS

Other objects, advantages and novel features of the present invention will become apparent from the following detailed description when considered in conjunction with the accompanying drawings.

FIG. 1 is a side, cross-sectional view of an ocular prosthesis of the present invention, in one embodiment.

FIG. 2 is a flow chart showing steps for practicing the method of the present invention, in one embodiment.

FIG. 3 is a cross-sectional view of a mold as may be used in forming the ocular prosthesis of FIG. 1.

FIG. 4 is a cross-sectional view of the posterior portion of the mold of FIG. 3. A layer of coring material has been laid into the posterior portion of the mold.

FIG. 5 is an exploded cross-sectional view of the mold of FIG. 3. The coring material of FIG. 4 has been removed, and a clear silicone-based material has been poured into the posterior portion of the mold. A sciera portion of the ocular prosthesis of FIG. 1 is ready to be laid into the mold.

FIG. 6 is a perspective view of a patient who has undergone an ocular resection or removal procedure. A molding material is being injected into the patient's orbit.

### DESCRIPTION OF CERTAIN EMBODIMENTS

The present invention provides a method for making an ocular prosthesis in which the prosthesis is fabricated from a non-toxic, biocompatible, silicone material. Prosthesis- or medical grade silicone is used as a base for the ocular prosthesis. The silicone material offers a soft and flexible alternative to known hardened ocular prostheses, and avoids certain shortcomings of the PMMA-based devices. For example, it is believed that an ocular prosthesis fabricated from medical grade silicone materials is not as porous and so does not host microorganisms that might be harmful to the patient's surrounding ocular tissue. It is also believed that the fabrication methods of the present invention are faster than methods of making PMMA based ocular prosthesis due, in one aspect, to faster curing times.

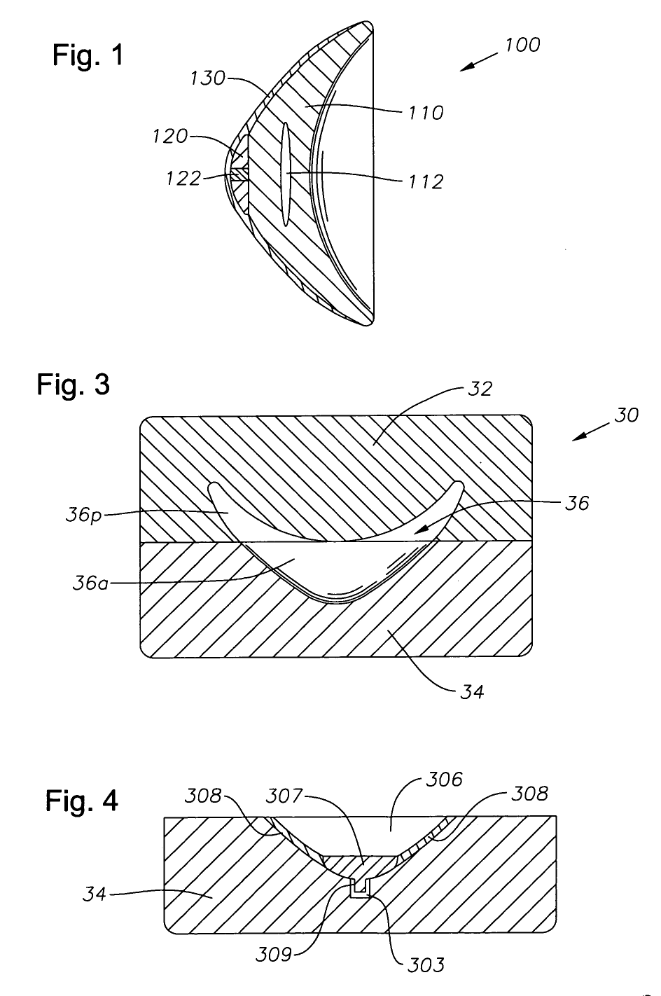

FIG. 1 presents a side, cross-sectional view of an ocular prosthesis 100 of the present invention, in one embodiment. The illustrative prosthesis 100 is intended to serve aesthetically as a substitute for a human eye. The prosthesis 100 is silicone-based, and is flexible.

The ocular prosthesis 100 first comprises a sclera portion 110. The sclera portion 110 represents what is commonly referred to in lay terms as the “white” of the eye. The sclera portion 110 is preferably fabricated from a medical grade silicone. Alternatively, the silicone material may be a prosthesis grade silicone, an implant grade silicone that is biocompatible over a period of time.

The medical grade silicone is white-tinted, and is cured following a molding process as will be described further below. The sclera portion 110 is preferably a solid device. However, as shown in FIG. 1, the sclera portion 110 may optionally include a hollow area 112. Use of a hollow portion 112 may further increase the flexibility and comfort of the ocular prosthesis 100, and potentially reduce cost due to the reduction in silicone materials.

The ocular prosthesis 100 also comprises an iris portion 120. The iris portion 120 is placed on the sclera portion 110. In one embodiment, the iris portion 120 is placed onto the sclera portion 110 manually by hand-painting. Preferably, silicone-base paints are used in order to create the appearance of a natural eye. In another embodiment, the iris portion 120 is created by printing a digital or other computer-based image in the appearance of a natural iris. In either embodiment, silicone-based paints may also be used to paint veins and other ocular features around the iris portion 120. In some processes, red cotton threading is mixed into the paint to create an even more natural appearance of the eye around the iris portion 120 as will be described further below.

In one aspect, the iris portion 120 and the sclera portion 110 are integrally formed. In other words, these two portions are not structurally distinct and are not formed via lamination or adherence. In this respect, the iris portion is preferably painted onto the sclera portion. However, the iris portion may be a printed disc that is adhered to the sciera portion 110. Alternatively, the iris portion 120 may be a painted acetate or acrylic disc that is laminated or adhered to the sclera portion 110.

The iris portion 120 further comprises a centrally-located pupil portion 122. The pupil portion 122 may be formed by creating a cylindrical depression within an annular region of the iris portion 120. The depression is then filled with a black-tinted silicone material.

According to one embodiment of the present invention, the ocular prosthesis 100 further comprises a transparent layer 130 of silicone material covering the sclera portion 110 and the surrounding iris portion 110. In this way a protective coating is provided over the ocular prosthesis 100 and iris portion 120. The transparent layer 130 also provides depth to the prosthesis 100.

Finally, a clear and glossy finish is preferably provided over the ocular prosthesis 100. In one aspect, a clear layer of silicone 130 is brushed onto the prosthesis 100 at a thickness of 1/16 inches. The clear layer of silicone 130 substantially covers an outer surface of the prosthesis 100.

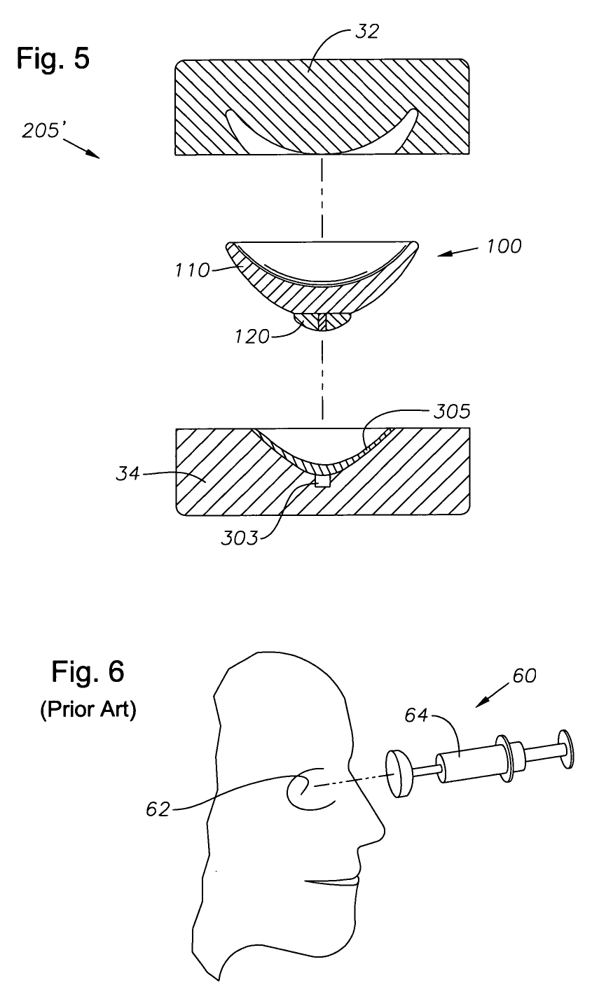

In order to form the ocular prosthesis, an impression of an eye socket of the patient is made. This may be in accordance with known prior art steps. FIG. 6 shows a perspective view of a patient 60. The patient has an eye socket 62 for holding the eye and corresponding muscles and nerves. However, this patient 60 has lost his eye due to either injury or disease. Thus, it is desirable to provide an ocular prosthesis for this patient 60.

In order to create an ocular prosthesis such as prosthesis 100, an impression is made of the patient's ocular socket 62. The impression is made using an impression material. The impression material may be an alginate. Alginate is a non-toxic, non-allergenic molding material. The impression material is placed into the patient's eye socket 62 using a syringe 64 or other injection device.

In some processes, dental gypsum is used to obtain a positive cast or mold from the impression. A wax material such as a dental base plate wax or inlay wax is then shaped onto the gypsum cast to achieve an empirical approximation of the anterior portion for the future prosthetic. This is known as a model or “master.” The master has the size and shape necessary for the prosthesis 100. The impression material typically solidifies to the consistency of the white of a boiled egg to form a custom master of the ocular prosthesis 100.

The master may be trimmed and further fitted to the patient's actual socket 62. The master may be modified in order to achieve patient 60 comfort. The master may also be modified for appropriate anterior/posterior dimensions, anterior curvature, eyelid symmetry, and iris center position. The master is also preferably fitted with a temporary iris disk. Indications of the proper placement and size of the iris and pupil are marked manually on the master in reference to the natural eye, and a corresponding hole is created on the master for the pupil. The master is then placed back into the patient's eye socket 62 to confirm the fit and orientation of the master as well as the disk. The master is then removed from the socket 62 and any final adjustments are made in the pliable wax material.

Once the master is finished, a two-part plaster mold is created from the master to generate the prosthesis 100. The two-part mold consists of a posterior portion and an anterior portion. The mold defines a shell or “negative.” The posterior and anterior portions may be clamped together during a molding process in accordance with existing technology.

FIG. 3 presents a side, cross-sectional view of an illustrative mold 30 as may be used in forming the ocular prosthesis 100 of FIG. 1. The mold 30 has a posterior portion 32 and a anterior portion 34. A cavity 36 is reserved from the master for receiving silicone-based setting materials that form the prosthesis 100. The cavity 36 has an anterior portion 36p (generally corresponding to the corneal, pupil and iris portion 120 of the prosthesis 100), and a posterior portion 36a (generally corresponding to the white, sclera portion 110 of the prosthesis 100).

A method for making an ocular prosthesis such as prosthesis 100 is also provided herein. The method uses a mold such as mold 30 for forming the prosthesis 100.

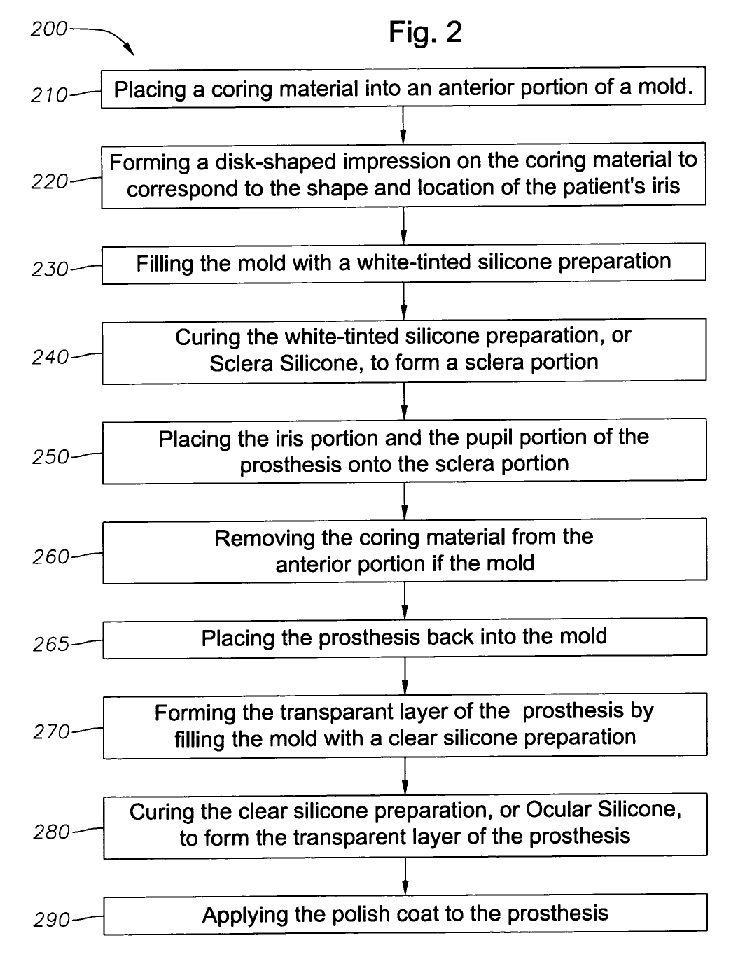

FIG. 2 presents a flow chart showing steps for performing the method 200, in one embodiment. The method 200 first includes the placement of a coring material 308 into the anterior portion 34 of the mold 30. This step is shown in step 210 of FIG. 2.

In one aspect, the coring material 208 is a layer of clay that is extruded into a thin sheet. A professional pasta maker may be used for the extrusion process. The clay is preferably sulfur free. The sulfur-free modeling clay is extruded at an even thickness and laid into the anterior portion 34 of the mold 30. Various clay thicknesses may be employed, though one preferred thickness is 1/16 of an inch. The clay is smoothed and contoured to create an even surface. The thin layer of clay creates a new, smooth surface inside the anterior cavity 36p. The layer of clay or “coring” material reserves a space that can later be used for a transparent layer 130.

FIG. 4 demonstrates the coring step 210. FIG. 4 is a cross-sectional view of the anterior portion 34 of the mold 30 of FIG. 3. The anterior portion 34 has a recess 306 which receives the silicone-based molding material that forms the sclera portion 110 of the prosthesis 100. A layer of coring material 308 has been laid into the recess 306 of the anterior portion 34.

Returning to FIG. 2, the method 200 also includes the step of forming a disk-shaped impression on the thin layer of clay 208. This is shown at step 220. In a preferred embodiment, the impression is created by placing a disk along the clay coring material 208 at the location corresponding to the iris portion 120.

FIG. 3 shows a disk 307 placed within the coring material 308. The disk 307 is preferably a plastic or acetate disk. The disk 307 is sized to correspond to the size of the iris portion 120, and the location corresponds to the location of the iris portion 120 in the final prosthesis 100. Use of the disk 307 creates a substantially flat, circular contour within the coring material 308. In one aspect, a central portion of the clay 308 is removed, and then replaced with the disk 307. In another aspect, the disk 307 extends into the clay either flush with the clay 308 or to create a recessed surface. The disk 307 preferably has a stem 309. In one aspect, the stem 309 is aligned with a pre-drilled hole 303 formed in the anterior portion 34 of the mold 30.

Next, the sclera portion 110 of the ocular prosthesis 100 is formed. This is done by filling the mold 30 with a white-tinted silicone preparation. This is demonstrated by step 230 of FIG. 2. Prior to the filling step 230, the posterior 32 and anterior 34 parts of the mold 30 may be sprayed with an acrylic coating such as Crystal Clear, and allowed to dry. This will seal the inner mold surfaces and prevent bubbles when the mold 30 is heated by release of hydrogen gas.

The white-tinted silicone preparation is preferably a custom-blended, medical grade, silicone material. The silicone material is used to form the body or sclera of the prosthesis 100, and may be referred to herein as “Sclera Silicone”. Originally, the Sclera Silicone is a translucent platinum material. However, it is tinted white using silicone pigments suspended in a silicone oil. A catalyst is preferably added to enable curing. The silicone oil may be, for example, a 50-centistoke to a 1,000-centistoke oil. The silicone material forming the Sclera Silicon may be specially ordered from a supplier, or may be mixed by the ocularist in the lab.

The durometer or firmness of the white-tinted silicone preparation can be adjusted with the addition of silicone diluent. Silicone diluent is a silicone oil that occupies space between the silicone molecules, extending the polymer chain and softening the final product.

The Sclera Silicone is optionally placed in a vacuum to remove any air bubbles. The white-tinted silicone material is then poured into the posterior portion 32 of the mold 30. A small amount of Sclera Silicone may also be placed in the anterior portion 34 of the mold 30 against the modeling clay 308. The mold 30 is then clamped together. The white-tinted silicone preparation, or Sclera Silicone, fills the recess 36. However, the white-tinted silicone preparation does not completely fill the recess 36 due to the presence of the coring material 308 along the surface of the anterior portion 34 of the mold 30.

The white-tinted silicone preparation is held in the mold 30 for a period of time. This allows the silicone preparation to cure to form the sclera portion 110. Box 240 of FIG. 2 shows the curing step. The silicone material in the mold 30 is preferably heat cured by placing the mold 30 in an oven. In one aspect, the oven is set at 150 to 175 degrees Fahrenheit. The Sclera Silicone is heated within the mold for about 5 to 50 minutes.

After the white-tinted silicone preparation has cured, the posterior portion 32 of the mold 30 is removed from the anterior portion 34. This exposes the newly cured sclera portion 110. The sclera portion 110 may then be removed from the mold 30.

After a suitable period of curing, the clamp is released and the mold 30 is pried open. The cured Sclera Silicone casting is removed from the mold 30, and is then cleaned with a solvent. The solvent helps to ensure bonding of a clear layer of silicone to be applied later. The cured Sclera Silicone forms the sclera portion 110 for the prosthetic 100. The sclera portion 110 may also be trimmed and otherwise prepped for painting.

A next step in the method 200, in one embodiment, is the placement of the iris portion 120 and pupil portion 122 on the sclera portion 110. This is represented at box 250 of FIG. 2. As noted, iris portion 120 may be manually painted to create the appearance of a natural eye. Painting is preferably done using silicone-based ink or other preparations with suitable, no-fade pigments. The ocularist may manually paint a disk using the silicone paints directly onto the newly-molded silicone sclera 110. This silicone painting material preferably has a thixotropic chemical added, and is tinted with selected silicone pigments in order to provide the desired color. In one aspect, the pigments are composed of oil pigment dispersed in a 50 centistoke silicone oil. Each paint pass may be instantly set (semi-cured) by spraying a custom solution of pure platinum in a solvent onto the painted surface. This allows the ocularist to add additional detail over a previously painted area without disturbing the painted detail underneath. Alternatively, to accommodate a more traditional ocularist, the ocularist may paint with traditional acrylic paints on an acetate disk which is then adhered to the silicone base.

When a silicone based ink is used, it is preferably set or semi-cured instantly with a thin layer of catalyst solution for curing the silicone paint. In a more preferred method, the sclera portion 110 or the iris portion 120 or both are painted multiple times for vivid details and effects, and each time the painting is semi-cured before a subsequent painting. Semi-curing before the next painting step allows the addition of more details without disturbing the painted details underneath. Other techniques, such as using cotton or silk fibers to add the effects of blood veins found in the natural eye, may also be used.

As noted, the iris portion 120 further comprises a centrally-located pupil portion 122. The pupil portion 122 may be formed by creating a cylindrical depression within an annular region of the iris portion 120. This may be done, for example, by using a biopsy punch (not shown) that corresponds in size to the patient's natural pupil. Disposable biopsy punches are available in different diameters, and may be used to punch out the pupil portion 122 at the center of the preserved circular annulus in the iris portion 120. The depression is then filled with a black-tinted silicone paint to create the pupil portion 122. This is preferably accomplished by using a fine brush. Once the depression is filled with the black-tinted silicone, the pupil portion 122 may be set with a platinum spray.

The placement of the iris portion 120 and the pupil portion 122 may alternatively include the use of digital photography and printing to form an iris that matches the “natural” eye of the patient. In this respect, the ocularist will use a software program such as Adobe Photoshop to create the iris and to otherwise form a digitally painted disk. This enables the ocularist to manipulate colors and details so as to accurately match the patient's natural eye. The digital file may be saved for future use or modification. This enables the ocularist to generate multiple iris disks with very little effort, and to further create a library of disks.

In this alternative digital embodiment, the iris portion 120 is created by printing a digital image in the shape of a disk and in the appearance of a natural iris. The iris disk is printed onto a durable substrate such as photo paper. The paper is sprayed with a fixative to prevent any smudging or change in color during fabrication of the prosthetic 100. The iris disk is cut out using either a punch or small scissors. The disk is then compared to the patient's natural eye for approval by the patient. Any necessary adjustments in size or color can be accomplished by manipulating the file on computer and reprinting the disk. The substrate with the approved disk is then adhered to the silicone sclera portion 110. The final printed iris disk may be secured to the sclera portion 110 with a small drop of one component silicone medical adhesive.

Next, the thin layer of clay 208 is removed from the anterior portion 34 of the mold 30. This step is shown at box 260 of FIG. 2. At the same time, the sclera portion 110 with the iris portion 120 is placed into the posterior portion 32 of the mold 30. This step is shown at box 265. The mold 30 is now prepared for the formation of a transparent layer.

FIG. 5 is an exploded cross-sectional view of the mold of FIG. 3. The coring material of FIG. 4 has been removed, and a clear silicone-based preparation 305 has been poured into the anterior portion 34 of the mold 30. The clear silicone-based preparation 305 will form a transparent layer 130, as seen in FIG. 1. The clear silicone-based preparation 305 is shown rounded in concave form as it will exist after the prosthetic 100 is inserted into the anterior portion 34 and the preparation 305 cures. However, in actuality it will form a level “puddle” that will conform to the void left by the coring material 308 once the mold 30 is clamped. The sclera portion 110 of the ocular prosthesis 100 of FIG. 1 is ready to be laid into the mold 30. The bore 303 in the anterior portion 34 has been filled.

The next step is the formation of the transparent layer 130. This is represented by box 270 of FIG. 2. The step of box 270 is performed by filling the anterior portion 34 of the mold 30 with a clear silicone preparation. A small amount of optically clear medical grade silicone (“Optical Silicone”) is mixed and catalyzed. In one aspect, the Optical Silicone is an optically clear platinum silicone. The Optical Silicone may be placed in a vacuum to remove air bubbles. This base silicone can be tinted with intrinsic pigment and painted over the surface of the sclera portion 110 to add detail. To create veins, a thin layer of the same Optical Silicone may be painted over the sclera portion 110, with pieces of red cotton fibers or threads positioned over the transparent layer 130 of the wet silicone. To cure the surface details, platinum solution may be sprayed over the surface, forcing an instant cure without the application of heat.

It is noted that the compositional difference between the Sclera Silicone and the Optical Silicone is clarity, strength, and durometer (firmness). The “Sclera Silicone” is translucent, not optically clear, and is firmer and more viscous than the Optical Silicone.

The “Optical Silicone” is preferably poured into the anterior side 34 of the mold 30, then the posterior side 32. The prosthesis 100 with painted sclera 110 and iris 120 portions is carefully submerged into the “Optical Silicone” and clamped in place. The mold 30 is then heat cured for about 5 to 50 minutes in an oven set at 150 to 175 degrees Fahrenheit. The curing step is shown at box 280 of FIG. 2.

After curing, the mold 30 is opened and the prosthesis 100 is removed. The prosthesis 100 now comprises the sclera portion 110, the iris portion 120, and the transparent layer 130. The transparent layer 130 allows the color patterns of the ocular prosthesis 100 to be under the protection of a transparent silicone coating. This helps protects the painted iris portion 120 and prevent the colors from fading or changing. This, in turn, allows the ocular prosthesis 100 to be more stable, and longer lasting.

The prosthesis 100 may be trimmed and buffed using an electric hand tool with special disks designed for contouring silicone. At this point, the transparent layer 130 of the ocular prosthesis 100 does not necessarily have a shiny or glossy finish. Therefore, it is preferred that a polish coat be applied to the exterior of the transparent layer 130 in order to create a glossy and transparent appearance. This step is shown at box 290 of FIG. 2.

Of interest, the method 200 of the present invention primarily employs an additive polishing technique. The unpolished prosthesis 100 is pushed onto a custom pin rig that impales the prosthesis 100 on a posterior side, providing a stable support with complete access to all surfaces of the silicone prosthesis 100. A small batch of catalyzed “Optical Silicone” with a platinum chemical additive is mixed creating the “Polish Coat.” This “Polish Coat” is applied sparingly to the outer surface (or transparent layer 130) of the prosthesis 100 until the prosthesis 100 resembles clear glass. The prosthesis 100 is allowed to air cure for about a half an hour to an hour, and is then heat cured for an additional hour in an oven set at 150 degrees Fahrenheit. Once cured, the silicone prosthesis 100 is complete and ready to be inserted into the patient's socket 62.

In some instances, a patient may already be in possession of a pre-fitted ocular prosthetic. The ocularist may choose to use the existing prosthetic to form the mold 30, rather than creating a new master. Thus, the present methods accommodate the use of an existing prosthetic. After the new mold 30 is formed, the steps outlined above may be employed for forming the silicone-based prosthetic 100.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. Furthermore, the teachings and disclosures of all references cited herein are expressly incorporated in their entireties by reference.

What is claimed is:

1. A method for making an ocular prosthesis for a patient, comprising:
filling a mold with a substantially white-tinted silicone material;
allowing the silicone material to cure to form a sciera portion of the ocular prosthesis; and
placing an iris portion onto the sclera portion to form the ocular prosthesis.

2. The method of claim 1, wherein the patient is a human.

3. The method of claim 1, further comprising inserting the ocular prosthesis into the eye socket of a patient.

4. The method of claim 1, wherein the white-tinted silicone material comprises a pigment suspended in a silicone oil.

5. The method of claim 4, wherein the silicone oil is a 50-centistoke to 1,000-centistoke oil.

6. The method of claim 1, wherein the white-tinted silicone material is a medical grade silicone.

7. The method of claim 1, wherein the white-tinted silicone material is a prosthesis grade or an implant grade silicone.

8. The method of claim 1, wherein the step of placing an iris portion onto the sclera portion comprises manually painting an iris with a pupil onto the iris portion.

9. The method of claim 1, wherein the step of placing an iris portion onto the sclera portion comprises adhering a colored disk onto the sclera portion.

10. The method of claim 9, wherein the colored disk is a printout of a digital image of an iris having a pupil.

11. The method of claim 3, further comprising:
applying a polish coat to the ocular prosthesis to create a glossy and transparent appearance before inserting the ocular prosthesis into the eye socket of a patient.

12. The method of claim 2, wherein the mold is prepared by:
making an impression of the eye socket of the patient to form a master; and
preparing the mold based on the master.

13. The method of claim 2, wherein the mold is prepared by:
taking a previously existing ocular prosthesis of the patient; and
preparing the mold based on the previously existing ocular prosthesis.

14. The method of claim 12, wherein the mold comprises:
a posterior portion corresponding to the sclera portion of the prosthesis; and
an anterior portion corresponding to the iris portion of the prosthesis.

15. The method of claim 14, further comprising:
placing a thin layer of coring material within the anterior portion of the mold in order to reserve a surface area of the sclera portion of the prosthesis.

16. The method of claim 15, wherein the coring material is a sulfur-free material.

17. The method of claim 16, wherein the coring material is clay.

18. The method of claim 16, wherein the coring material is wax.

19. The method of claim 12, further comprising:
removing a central portion of the coring material; and
inserting a disk into the central portion of the coring material, thereby forming a disk-shaped impression on the thin layer of coring material at a location corresponding to the iris portion to be placed on the sclera portion, wherein the size of the disk-shaped impression corresponds to the size of an iris.

20. The method of claim 19, further comprising:
filling the disk-shaped impression with the substantially white-tinted silicone material to add to the sclera portion of the ocular prosthesis, and thereby creating an elevated disk.

21. The method of claim 19, further comprising:
removing a center portion of the disk to create a depression that corresponds to the size and shape of a pupil in the iris portion.

22. The method of claim 21, wherein the step of placing an iris portion onto the sclera portion further comprises filling the depression with black silicone paint to create the pupil in the iris portion.

23. The method of claim 11, wherein the step of placing an iris portion onto the sclera portion comprises manually painting the iris portion around the pupil prior to applying the polish coat.

24. The method of claim 23, wherein painting the iris portion around the pupil is done using silicone-based paints.

25. The method according to claim 24, wherein after the step of painting the iris portion around the pupil, the paint is at least partially cured with a catalyst solution for curing the silicone paint.

26. The method of claim 15, further comprising:
removing the thin layer of clay from the posterior portion of the mold;
placing the ocular prosthesis into the posterior portion of the mold;
at least partially filling the mold with an optically clear silicone preparation;
closing the mold; and
allowing the clear silicone preparation to cure to form a transparent layer.

27. The method of claim 26, further comprising:
removing the ocular prosthesis from the mold; and
applying a polish coat to the ocular prosthesis to create a glossy and transparent appearance.

28. The method of claim 26, further comprising:
prior to the step of filling the mold with a clear silicone preparation, painting the sclera portion, the iris portion, or both, to create natural details for the ocular prosthesis.

29. The method of claim 28, wherein the natural details comprise at least one of blood vessels, the limbus, and pink-shaded corner areas.

30. The method of claim 26, further comprising:
removing surface irregularities from the cured silicone preparation of the ocular prosthesis.

31. The method of claim 26, wherein the step of allowing the clear silicone preparation to cure is performed at an elevated temperature.

32. The method of claim 1, further comprising:
placing a vacuum on the substantially white-tinted silicone material to remove air bubbles prior to filling the mold.

33. The method of claim 1, wherein the sclera portion is solid.

34. The method of claim 1, wherein the sclera portion has a hollow portion.

35. An ocular prosthesis, comprising:
a posterior sclera portion fabricated from a white-tinted silicone material and formed from a molding process;
an anterior iris portion placed on the sclera portion; and
a clear coat finish over the sclera portion and the iris portion to provide a glossy finish.

36. The ocular prosthesis of claim 35, further comprising a transparent layer between the iris portion and the clear coat finish.

37. The ocular prosthesis of claim 36, wherein:
the iris portion is manually painted using silicone-based paints to create an appearance of a natural eye; and
the iris portion further comprises a centrally-located pupil portion formed from a depression in an annular portion of the iris, the depression being filled with a black-tinted silicone material.

38. The ocular prosthesis of claim 37, wherein the iris portion is created by printing a digital image in the appearance of a natural iris.

39. The ocular prosthesis of claim 37, wherein the clear coat finish and the transparent layer are each fabricated from silicone-based materials.
